(19) United States
(12) Patent Application Publication
(10) Pub. No.: US 2005/0085916 A1
(43) Pub. Date: Apr. 21, 2005

(54) Apparatus and method for replacing the nucleus pulposus of an intervertebral disc or for replacing an entire intervertebral disc

(76) Inventors: Lehmann K. Li, Milford, CT (US); Stephen A. Maguire, Huntington, CT (US); Edward F. Leonard, Bronxville, NY (US); Ernest N. Corrao JR., Bethel, CT (US); Robert Stanton Ward, Lafayette, CA (US); Sallie Kate Coviello, San Francisco, CA (US)

Correspondence Address: WOODARD, EMHARDT, MORIARTY, MCNETT & HENRY LLP;BANK ONE TOWER/CENTER, 111 MONUMENT CIRCLE, SUITE 3700, INDIANAPOLIS, IN, 46204-5137, US

(21) Appl. No.: 10/974,662

(22) Filed: Oct. 27, 2004

Related U.S. Application Data
(63) Continuation of application No. 10/011,916, filed on Nov. 5, 2001.
(63) Continuation-in-part of application No. 09/559,899, filed on Apr. 26, 2000, now Pat. No. 6,764,514.

(51) Int. Cl. A61F 2/44

(52) U.S. Cl. 623/17.16; 623/17.11

(57) ABSTRACT

A prosthetic nucleus pulposus for replacing the natural nucleus pulposus of an intervertebral disc. The prosthetic nucleus proposus comprises a partially collapsed sealed envelope formed from a material which is permeable to extracellular body fluid. The envelope contains a solute which provides an osmotic potential across the walls of the envelope. In use, the partially collapsed envelope is surgically implanted in the hallowed-out interior of an intervertebral disc and is allowed to absorb fluid, whereby expansion of the envelope and subsequent disc expansion is accomplished.

### REFERENCE TO PENDING PRIOR PATENT APPLICATION

This patent application claims benefit of pending prior U.S. patent application Ser. No. 09/559,899, filed Apr. 26, 2000 by Lehmann K. Li et al. for PROSTHETIC APPARATUS AND METHOD (Attorney's Docket No. LMT-62), which patent application is hereby incorporated herein by reference.

### FIELD OF THE INVENTION

This invention relates to surgical apparatus and methods in general, and more particularly to surgical apparatus and methods for the repair and/or replacement of the nucleus pulposus of an intervertebral disc or for the replacement of an entire intervertebral disc.

### BACKGROUND OF THE INVENTION

The spinal column is a flexible chain of closely linked vertebral bodies. In a normal human spine, there are seven cervical, twelve thoracic and five lumbar vertebral bodies. Below the lumbar vertebrae are the sacrum and coccyx. Each individual vertebral body has an outer shell of hard, dense bone. Inside the vertebral body is a honeycomb of cancellous bone containing red bone marrow. All of the red blood cells, and many of the white blood cells, are generated inside such cancellous bone, where the blood cells mature before being released into the blood stream.

The intervertebral disc, which is also known as the spinal disc, serves as a cushion between the vertebral bodies so as to permit controlled motion. A healthy intervertebral disc consists of three components: a gelatinous inner core called the nucleus pulposus (or, more simply, the nucleus); a series of overlapping and laminated plies of tough fibrous rings called the annulus fibrosus (or, more simply, the annulus); and two (i.e., superior and inferior) thin cartilage layers, connecting the intervertebral disc to the thin cortical bone of the adjacent vertebral bodies, called the end plates.

An intervertebral disc may be displaced and/or damaged due to trauma (such as a herniated disc), or disease (such as a degenerative disc disease).

A herniated disc may bulge out and compress itself onto a nerve, resulting in lower leg pain, loss of muscle control or paralysis. To treat a herniated disc, the offending portions of the disc (i.e., the bulging portions of the nucleus) are generally removed surgically.

A degenerative disc disease typically causes the disc to gradually reduce in height, causing the annulus to buckle, tear or separate, radially and/or circumferentially, and causing persistent and disabling back pain. Degenerative disc disease is generally treated by surgically removing the nucleus and fusing together the adjacent vertebral bodies so as to stabilize the joint.

In either case, whether removing some or all of the nucleus, these procedures ultimately place greater stress on adjacent discs due to their need to compensate for the lack of motion. This may in turn cause premature degeneration of those adjacent discs.

Modern trends in surgery include the restoration, rather than the removal, of anatomical structures, with this restoration preferably being effected through the use of minimally invasive surgical techniques. The ability to surgically repair damaged tissues or joints, creating as few and as small incisions as possible, generally produces less trauma and pain for the patient while yielding better clinical outcomes.

In this respect it has been recognized that it may be possible to replace a damaged nucleus pulposus with a prosthetic implant, whereby to restore the spinal disc to its original configuration and function. Unfortunately, however, such implants, sometimes referred to as a “prosthetic nucleus”, tend to suffer from a variety of deficiencies.

For one thing, the natural nucleus is a sophisticated structure which is difficult to reproduce artificially. It must carry a wide range of different loads, depending on the individual's current activity. By way of example, the nucleus must carry a relatively large load while the individual is carrying a heavy object, yet must accommodate a relatively modest load while the individual is lying down (e.g., sleeping). Furthermore, the nucleus must be able to respond quickly to rapidly changing loads (e.g., while the individual is jumping up and down). The natural nucleus accommodates such load changes by means of an appropriate controlled deformation.

A prosthetic nucleus which does not adequately deform with changing loads (i.e., one which is inadequately compliant) is unable to properly absorb shock loads in the spine and thus is unlikely to emulate the shock response of the natural nucleus. On the other hand, a prosthetic nucleus that expands and contracts excessively under sustained changes in load (i.e., one which is excessively compliant) is likely to cause undesirable anatomical changes involving the vertebrae, the spinal nerves and other adjacent structures. Again, such a prosthetic nucleus is not likely to emulate the response of the natural nucleus.

A capacity to provide an appropriate deformational response to different loadings is therefore highly desirable in a prosthetic nucleus. Unfortunately, current prosthetic nuclei have difficulty reproducing the variable load-carrying capability of the natural nucleus.

Another deficiency of current prosthetic nuclei is that they generally require relatively large or multiple incisions in the annulus in order to insert the prosthetic nucleus into the interior of the spinal disc. Such large or multiple incisions tend to further weaken an already compromised disc. Additionally, these incisions in the annulus are generally not easily repaired; thus, there can be a concern that the prosthetic nucleus may eventually work its way back out of the disc space and interfere with the surrounding anatomy.

A further deficiency of current, less-invasive prosthetic nuclei (see, for example, U.S. Pat. No. 5,674,295, issued Oct. 7, 1997 to Ray et al.) is that multiple, laterally-spaced implants typically have to be used to recreate the nucleus, which suggests that the side-by-side positioning of the several implants has to be carefully considered so as to ensure proper carrying of the load.

In addition to the foregoing, it should also be appreciated that an inability to properly control the deformation of a prosthetic nucleus consequent to different loadings may also result in the transmission of high radial stresses to the annulus, which may already have been compromised by trauma and/or disease, and is in any case compromised by the incisions required for insertion of the prosthetic nucleus.

Replacement of the entire intervertebral disc has also been proposed. However, such prosthetic intervertebral discs are also believed to suffer from the load-carrying issues discussed above with respect to prosthetic nuclei.

### SUMMARY OF THE INVENTION

Accordingly, one object of the present invention is to provide improved apparatus for replacing the nucleus pulposus of an intervertebral disc.

Another object of the present invention is to provide an improved method for replacing the nucleus pulposus of an intervertebral disc.

And another object of the present invention is to provide improved apparatus for replacing an entire intervertebral disc.

Still another object of the present invention is to provide an improved method for replacing an entire intervertebral disc.

With the above and other objects in view, a feature of the present invention is the provision of a novel prosthetic nucleus pulposus for replacing the natural nucleus pulposus of an intervertebral disc, wherein the prosthetic nucleus pulposus comprises a closed envelope comprising a membrane and containing at least one solute therein, wherein the membrane is permeable to water and impermeable to the at least one solute, and wherein the at least one solute is soluble in water, whereby when the closed envelope is deployed in an environment containing water, the water will pass through the membrane, contacting the at least one solute and causing the at least one solute to go into solution, thereby establishing an osmotic engine by which the envelope will inflate and pressurize. This inflation will continue until an equilibrium condition is established between the internal and external pressures acting on the envelope. In accordance with the present invention, the closed envelope comprises a construction and the at least one solute comprises a material and a quantity sufficient to generate an internal pressure, when the prosthetic nucleus pulposus is deployed in the body, which is (1) significantly greater than the external pressure imposed on the prosthetic nucleus pulposus by external forces, with the closed envelope being capable of withstanding such internal pressure, with the volume of the prosthetic nucleus pulposus remaining relatively constant even as the external load imposed on the prosthetic nucleus pulposus changes, and (2) low enough that the prosthetic nucleus pulposus will remain adequately compliant to changing external loads by accommodating changing external loads in the short term by an appropriate controlled deformation of the closed envelope.

Another feature of the present invention is the provision of a novel method for replacing the nucleus pulposus of an intervertebral disc, wherein the method comprises the steps of:
providing a prosthetic nucleus pulposus comprising a closed envelope comprising a membrane and containing at least one solute therein, wherein the membrane is permeable to water and impermeable to the at least one solute, and wherein the at least one solute is soluble in water, whereby when the closed envelope is deployed in an environment containing water, the water will pass through the membrane, contacting the at least one solute and causing the at least one solute to go into solution, thereby establishing an osmotic engine by which the envelope will inflate and pressurize, with this inflation continuing until an equilibrium condition is established between the internal and external pressures acting on the envelope, and further wherein the closed envelope comprises a construction and the at least one solute comprises a material and a quantity sufficient to generate an internal pressure, when the prosthetic nucleus pulposus is deployed in the body, which is (1) significantly greater than the external pressure imposed on the prosthetic nucleus pulposus by external forces, with the closed envelope being capable of withstanding such internal pressure, with the volume of the prosthetic nucleus pulposus remaining relatively constant even as the external load imposed on the prosthetic nucleus pulposus changes, and (2) low enough so that the prosthetic nucleus pulposus will remain adequately compliant to changing external loads by accommodating changing external loads in the short term by an appropriate controlled deformation of the closed envelope;
creating a void in the natural nucleus pulposus of an intervertebral disc; and
deploying the prosthetic nucleus pulposus in the void in the intervertebral disc.

A further feature of the present invention is the provision of a novel prosthetic intervertebral disc, wherein the prosthetic intervertebral disc comprises a closed envelope comprising a membrane and containing at least one solute therein, wherein the membrane is permeable to water and impermeable to the at least one solute, and wherein the at least one solute is soluble in water, whereby when the closed envelope is deployed in an environment containing water, water will pass through the membrane, contacting the at least one solute and causing the at least one solute to go into solution, thereby establishing an osmotic engine by which the envelope will inflate and pressurize. This inflation will continue until an equilibrium condition is established between the internal and external pressures acting on the envelope. In accordance with the present invention, the closed envelope comprises a construction and the at least one solute comprises a material and a quantity sufficient to generate an internal pressure, when the prosthetic intervertebral disc is deployed in the body, which is (1) significantly greater than the external pressure imposed on the prosthetic intervertebral disc by external forces, with the closed envelope being capable of withstanding such internal pressure, with the volume of the prosthetic intervertebral disc remaining relatively constant even as the external load imposed on the prosthetic intervertebral disc changes, and (2) low enough that the prosthetic intervertebral disc will remain adequately compliant to changing external loads by accommodating changing external loads in the short term by an appropriate controlled deformation of the closed envelope.

Another feature of the present invention is the provision of a novel method for replacing an intervertebral disc, wherein the method comprises the steps of:
providing a prosthetic intervertebral disc comprising a closed envelope comprising a membrane and containing at least one solute therein, wherein the membrane is permeable to water and impermeable to the at least one solute, and wherein the at least one solute is soluble in water, whereby when the closed envelope is deployed in an environment containing water, the water will pass through the membrane, contacting the at least one solute and causing the at least one solute to go into solution, thereby establishing an osmotic engine by which the envelope will inflate and pressurize, with this inflation continuing until an equilibrium condition is established between the internal and external pressures acting on the envelope, and further wherein the closed envelope comprises a construction and the at least one solute comprises a material and a quantity sufficient to generate an internal pressure, when the prosthetic intervertebral disc is deployed in the body, which is (1) significantly greater than the external pressure imposed on the prosthetic intervertebral disc by external forces, with the closed envelope being capable of withstanding such internal pressure, with the volume of the prosthetic intervertebral disc remaining relatively constant even as the external load imposed on the prosthetic intervertebral disc changes, and (2) low enough so that the prosthetic intervertebral disc will remain adequately compliant to changing external loads by accommodating changing external loads in the short term by an appropriate controlled deformation of the closed envelope;
removing the natural intervertebral disc; and
deploying the prosthetic intervertebral disc in the void left by the removal of the natural intervertebral disc.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts and further wherein:

FIG. 1 is a schematic side view of a novel prosthetic nucleus pulposus formed in accordance with the present invention, with the prosthetic nucleus pulposus being shown in a partially inflated condition;

FIGS. 2-5 are schematic side views similar to that of FIG. 1, but showing alternative constructions;

FIG. 6 is a schematic side view showing the prosthetic nucleus pulposus of FIG. 1 in an inflated condition;

FIG. 6A is a schematic diagram illustrating the force balance associated with the prosthetic nucleus pulposus (and prosthetic intervertebral disc) of the present invention;

FIG. 7 is a schematic side view showing the prosthetic nucleus pulposus of FIG. 1 deployed in a void created in a spinal disc;

FIG. 8 is a schematic side view showing an incision for inserting the prosthetic nucleus pulposus into the interior of the spinal disc;

FIG. 9 is a schematic view similar to that of FIG. 7, except showing the prosthetic nucleus pulposus in an inflated condition;

FIG. 10 is a schematic side view showing an alternative form of prosthetic nucleus pulposus;

FIGS. 11-14 are schematic views showing another alternative form of prosthetic nucleus pulposus;

FIG. 15 is a schematic top view showing still another alternative form of prosthetic nucleus pulposus;

FIG. 16 is a partial schematic perspective view showing another form of prosthetic nucleus pulposus formed in accordance with the present invention;

FIG. 17 is a schematic side view showing still another form of prosthetic nucleus pulposus formed in accordance with the present invention;

FIG. 17A is a schematic perspective view showing another form of prosthetic nucleus pulposus formed in accordance with the present invention;

FIG. 18 is a partial schematic perspective view showing yet another form of prosthetic nucleus pulposus formed in accordance with the present invention;

FIG. 19 is a schematic view illustrating the pressure-volume relationship of the prosthetic nucleus pulposus;

FIGS. 20-25 are schematic views illustrating a preferred technique for folding a prosthetic nucleus pulposus into a delivery cannula; and

FIG. 26 is a schematic, combined top and side view of a prosthetic nucleus formed in accordance with the present invention.

### DETAILED DESCRIPTION OF THE PREFERRED EMBODIMENTS

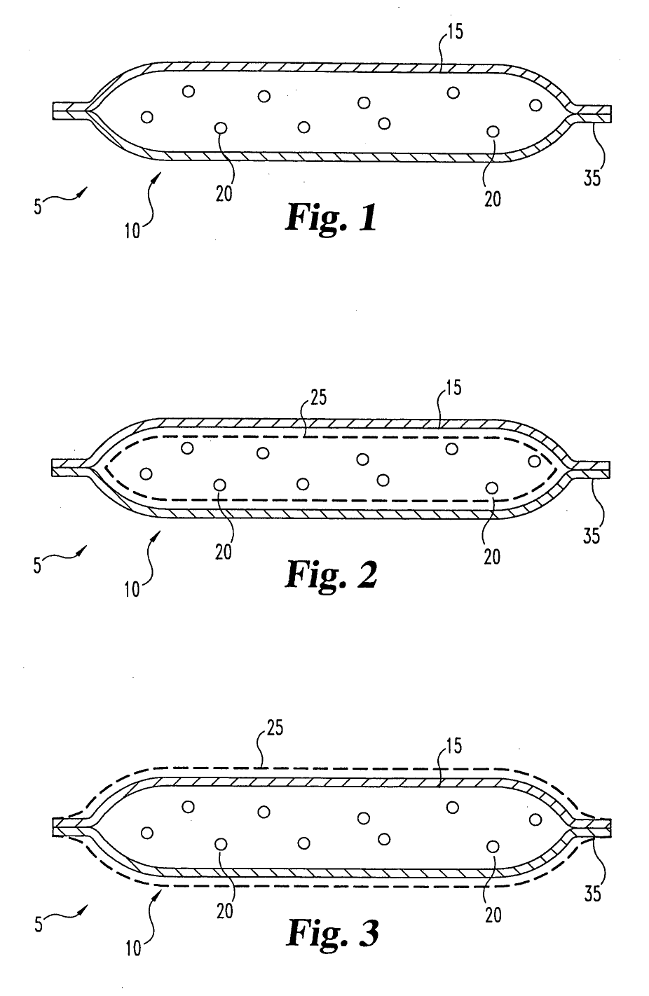

Looking first at FIG. 1, there is shown a prosthetic nucleus pulposus (or, more simply, prosthetic nucleus) 5. Prosthetic nucleus 5 generally comprises a closed envelope 10 which comprises a membrane 15 and which contains at least one solute 20 therein which provides an osmotic potential across membrane 15.

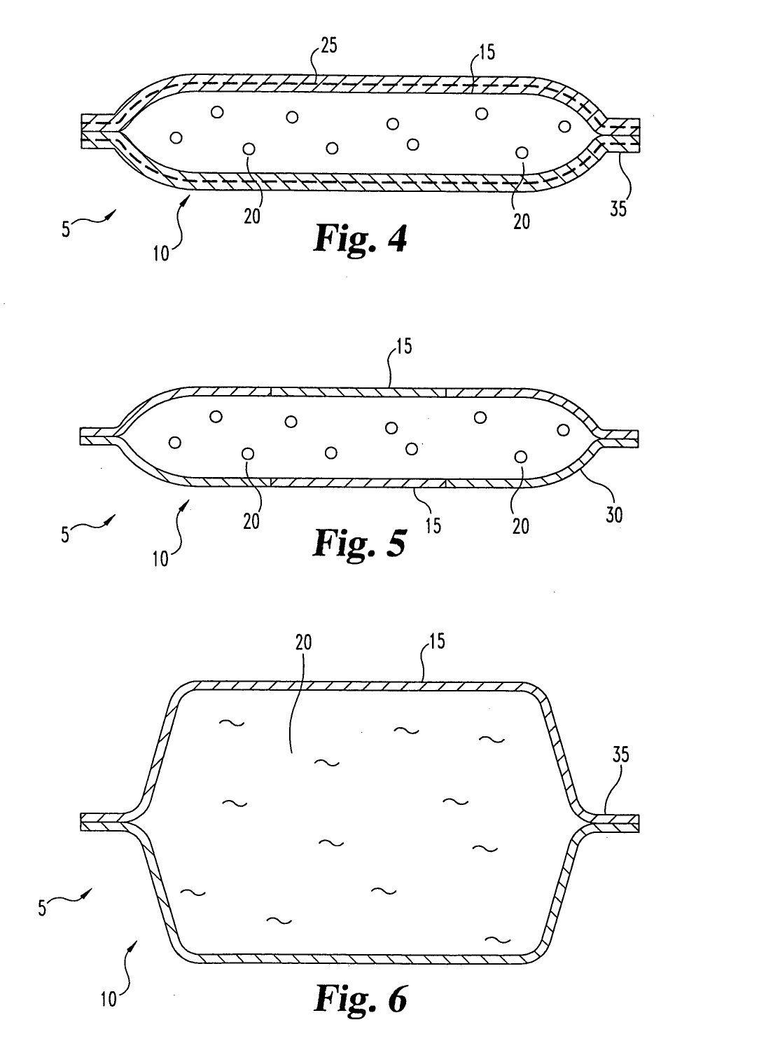

Closed envelope 10 can be formed substantially entirely out of membrane 15, such as is shown in FIG. 1, with or without an accompanying reinforcing structure, e.g., a supporting mesh 25 positioned internal to membrane 15 (FIG. 2) or external to membrane 15 (FIG. 3) or contained within membrane 15 (FIG. 4).

Alternatively, closed envelope 10 can be formed with some other construction incorporating membrane 15 therein, e.g., membrane 15 can comprise one or more windows formed in a wall 30 of envelope 10, such as is shown in FIG. 5.

In any case, closed envelope 10 comprises a closed structure captivating at least one solute 20 therein and including membrane 15 as a selective portal into closed envelope 10.

Membrane 15 is formed from one or more materials so as to be permeable to water and impermeable to the at least one solute 20 contained within closed envelope 10. As a result of this construction, when a solute soluble in water is placed inside closed envelope 10 and the closed envelope is deployed in an environment containing water, the water will pass through membrane 15, contacting the solute and causing the solute to go into solution, thereby establishing an osmotic engine by which the envelope will inflate and pressurize. This inflation will continue until an equilibrium condition is established between the internal and external pressures acting on the envelope.

More particularly, the present invention relies upon the following phenomena: water will move from one solution to another across a suitable membrane in a direction that is determined by the osmotic pressures of the two solutions and the hydrostatic pressures in the two solutions. Water will move into the solution whose difference of osmotic and hydrostatic pressures is greater than that difference in the other solution. Water will move at a rate that is generally proportional to the imbalance between the aforementioned pressure differences of the respective solutions. This imbalance between the respective solutions is commonly termed the osmotic driving force for water movement. Water movement will cease when the two pressure differences are equal and this condition is called osmotic equilibrium.

The osmotic pressure of a solution generally increases with the molar concentration of solute in the solution. Thus, if a suitable membrane in an envelope that resists expansion confines a solute, water will move into the envelope with the effects of decreasing the concentration of the solute within the envelope and raising the hydrostatic pressure of the solution in the envelope. Both of these effects serve to decrease the driving force for further water transport and their action will, if allowed to persist, result in osmotic equilibrium.

The application of a compressive mechanical force to the envelope will generally result in an increase of hydrostatic pressure within the envelope. This force may arise with the same effect if the envelope expands against an object that resists displacement, or if an object is forced against the envelope. This increase in hydrostatic pressure will change the equilibrium volume of the envelope. However, by establishing a system with relatively high internal pressure, such changes in the envelope's equilibrium volume can be kept relatively small, e.g., within anatomically—appropriate limits. With envelopes that respond to mechanical forces according to the direction and location of an applied force, changes in shape due to variations in the magnitude of applied mechanical forces will depend on the direction and location of such force. It is beneficial and possible to design envelopes with different responses in volume and shape to applied forces according to the direction of the force and the part of the surface of the envelope to which the force is applied.

This invention demonstrates the use of these phenomena to produce a prosthetic nucleus that will control the force between the nucleus and the surrounding annulus, while allowing a substantial and natural force to exist between the nucleus and contiguous vertebrae, with a small and suitable change in intervertebral distance over the range of spinal loads (forces) that are encountered during rest and physical activity.

By way of example but not limitation, membrane 15 may comprise a homogenous membrane with suitable water permeable characteristics. Membrane 15 may comprise polyurethane block copolymers with hydrophilic segments. Membrane 15 may comprise cellulose acetate, cellulose acetate butyrate, cellulose nitrate, crosslinked polyvinyl alcohol, polyurethanes, nylon 6, nylon 6.6, aromatic nylon, polyvinyl acetate, plasticized polyvinyl acetate, polyvinyl butyrate, and ethylene vinyl acetate copolymers.

In one preferred form of the invention, membrane 15 forms the entire envelope 10, and membrane 15 is formed out of polyurethane block copolymers with hydrophilic segments.

The thickness of membrane 15 can vary, depending on considerations such as (1) the material used to form membrane 15; (2) the overall size of membrane 15; (3) the desired membrane strength; and (4) the desired rate of osmotic flow. With respect to this latter consideration, it has been found that osmotic flow is generally substantially inversely proportional to membrane thickness.

In one preferred form of the invention, membrane 15 has a thickness of about 0.010 to 0.030 inch. This thickness is chosen to provide a reasonable balance between membrane strength and the rate of osmotic flow, and may change over the length of the membrane.

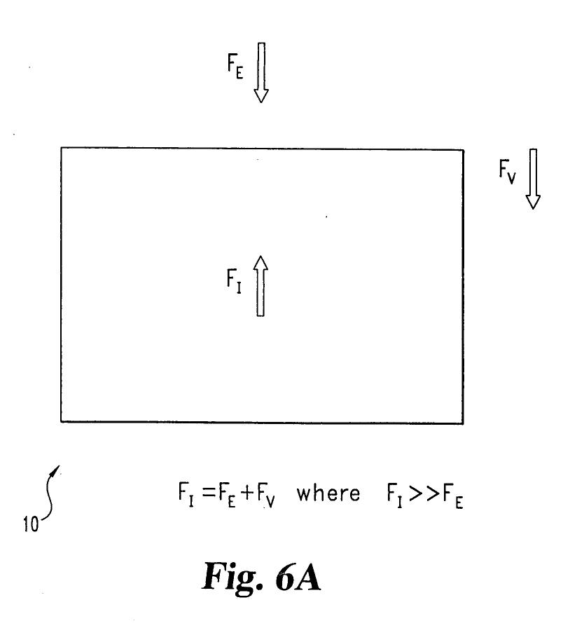

Inasmuch as prosthetic nucleus 5 must fit within a spinal disc, the shape of envelope 10 is generally significant. More particularly, and as will be discussed in further detail below, envelope 10 is shaped so that, upon expansion (FIG. 6), prosthetic nucleus 5 will assume a shape similar to the natural nucleus it is to replace.

In one preferred form of the invention, envelope 10 is configured so as to have a disc-like shape.

Envelope 10 is normally closed with a seal 35 (FIGS. 1 and 6) after the at least one solute 20 has been placed inside. As a result, the at least one solute 20 is captured within envelope 10, with water able to enter envelope 10 via membrane 15. Any suitable seal may be used to close off envelope 10, provided that the seal is capable of making a sufficiently fluid-tight closure so that water enters envelope 10 only through membrane 15. Seal 35 can be formed from the same material as membrane 15, or it can be formed from another material such as a sealant (e.g., glue).

In one preferred form of the invention, envelope 10 is sealed by heat sealing together opposing sections of the membrane material, such as is shown in FIGS. 1 and 6.

The at least one solute 20 can be any material or materials useful to establish the desired osmotic pressure across the membrane without degrading the membrane, and which is biocompatible. Such biocompatibility is important in case envelope 10 should leak or rupture after deployment in the body. The at least one solute 20 may be a solid (e.g., particles, powder, one or more tablets, etc.), a paste, a liquid concentrate, etc. The at least one solute 20 is preferably placed in envelope 10 prior to deploying prosthetic nucleus 5 in the body; however, solute 20 may also be placed in envelope 10 after prosthetic nucleus 5 has been deployed in the body, e.g., by using a syringe.

By way of example but not limitation, the at least one solute 20 may comprise polyacrylamide. The at least one solute may comprise one or more salts such as sodium chloride, calcium chloride, magnesium chloride, magnesium sulfate, potassium sulfate, potassium chloride, sodium sulfate, sodium acetate, ammonium phosphate, ammonium sulphate, calcium lactate or magnesium succinate. The at least one solute 20 may also comprise one or more non-ionic substances such as sucrose, glucose, fructose, glycine, alanine, valine and vinyl pyrrolidone. The at least one solute 20 may also comprise one or more hydrophilic (water or soluble) polymers such as poly-n-vinylpyrrolidone, carboxymethylcellulose and polyethylene glycols. The at least one solute 20 may also comprise manitol, urea, blood byproducts, proteins and dextran. Still other materials will be apparent to those skilled in the art in view of the present disclosure.

In one preferred form of the invention, the at least one solute 20 comprises polyacrylamide.

The at least one solute 20 comprises a material and a quantity sufficient to generate an internal pressure, when the prosthetic nucleus is deployed in the body, which is (1) significantly greater than the external pressure imposed on the prosthetic nucleus by external forces, with the closed envelope being capable of withstanding such internal pressure, with the volume of the prosthetic nucleus remaining relatively constant even as the external load imposed on the prosthetic nucleus changes, and (2) low enough that the prosthetic nucleus will remain adequately compliant to changing external loadsby accommodating changing external loads in the short term by an appropriate controlled deformation of the closed envelope.

More particularly, and looking now at FIG. 6A, there is shown a schematic diagram illustrating in simplified form the force balance associated with the prosthetic nucleus (and prosthetic intervertebral disc) of the present invention.

In general, it will be seen that where FE represents the external forces imposed on the prosthetic nucleus 5, F_{I} represents the internal forces generated inside envelope 10 due to pressures, and F_{V} represents the tensile forces induced in envelope 10,
F_{I}═F_{E}+F_{V}

In accordance with the present invention, the at least one solute 20 comprises a material and a quantity sufficient to generate, when the prosthetic nucleus is deployed in the body, F_{I}>>F_{E}. The volume of the prosthetic nucleus will remain relatively constant even as the external load on the prosthetic nucleus changes. At the same time, it is also important for F_{I} to be low enough that the prosthetic nucleus will remain adequately compliant to changing external loads, i.e., by accommodating changing external loads in the short term by an appropriate controlled deformation of the closed envelope.

It will be appreciated that inasmuch as F_{I}>>F_{E}, F_{V} will be a sizable force. In other words, the tensile forces induced in envelope 10 will be substantial. These tensile forces may be provided by membrane 15 itself (FIG. 1), and/or by membrane 15 in combination with supporting mesh 25 (FIGS. 2-4), and/or by membrane 15 in combination with wall 30 (FIG. 5), etc.

It is generally desirable that the prosthetic nucleus be small and flexible upon implantation and be provided with the ability to achieve a larger volume after it is in place. One component that determines the inital volume and flexibility of the prosthetic nucleus at the time of implantation is the solute volume. Inasmuch as osmotic pressure depends on the number of molecules present in a unit volume (i.e. the molar concentration), it is generally desirable to choose a solute with a small volume and weight per molecule. In dilute solutions, all solutes exert the same osmotic pressure at the same molar concentration and thus conform to van't Hoff's law. At higher concentrations, solutes can differ in the osmotic pressure they generate at a fixed molar concentration. It is preferable to utilize a solute that exhibits a positive deviation from van't Hoff's law and thus generates a higher osmotic pressure than that law predicts.

In general, high osmotic pressures may be achieved by the use of large weights of a solute in a given volume, or by the use of proportionately less weights of a solute of lesser molecular weight. At concentrations that produce usefully high osmotic pressures, a solute may produce osmotic pressures that follow the equation of van't Hoff or they may be “non-ideal”, producing pressures higher (positive deviation) or lower (negative deviation) than the equation predicts. In order to minimize insertion volume, the present invention is served by the choice of a low molecular weight, water-soluble solute that exhibits a strong positive deviation from van't Hoff's law. In its simplest embodiment, this invention utilizes a solute that is completely impermeable through the envelope so that the osmotic capability of the system remains constant over the lifetime of the implant. The choice of this solute and the membrane component of the envelope must thus be made together. In particular, solutes of small molecular weight will more easily penetrate most membranes that are permeable to water and might otherwise be chosen to embody this invention.

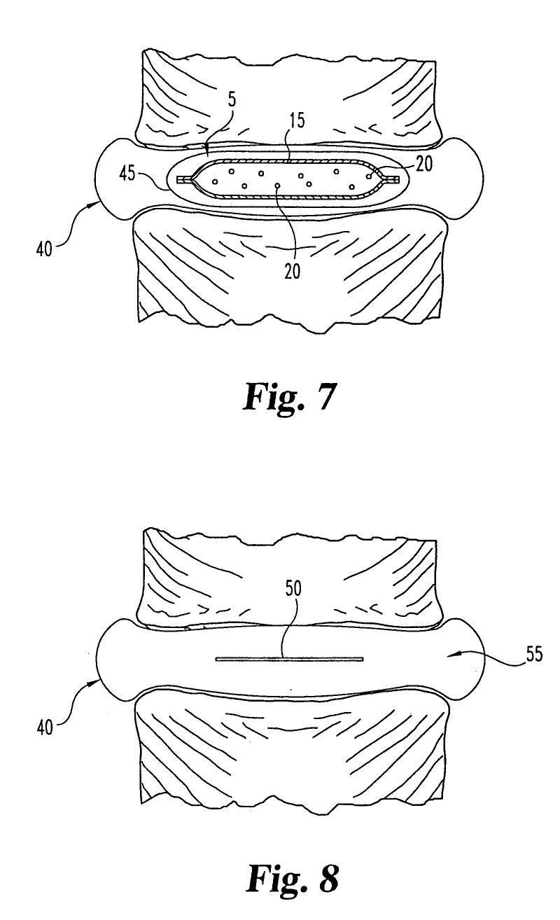

Referring now to FIG. 7, prosthetic nucleus 5 is shown surgically implanted into an intervertebral disc 40 which has had some or all of its natural nucleus removed so as to create a void 45 therein. Prosthetic nucleus 5 is preferably surgically implanted into the void 45 in a collapsed statethrough an incision 50 (FIG. 8) formed in annulus 55.

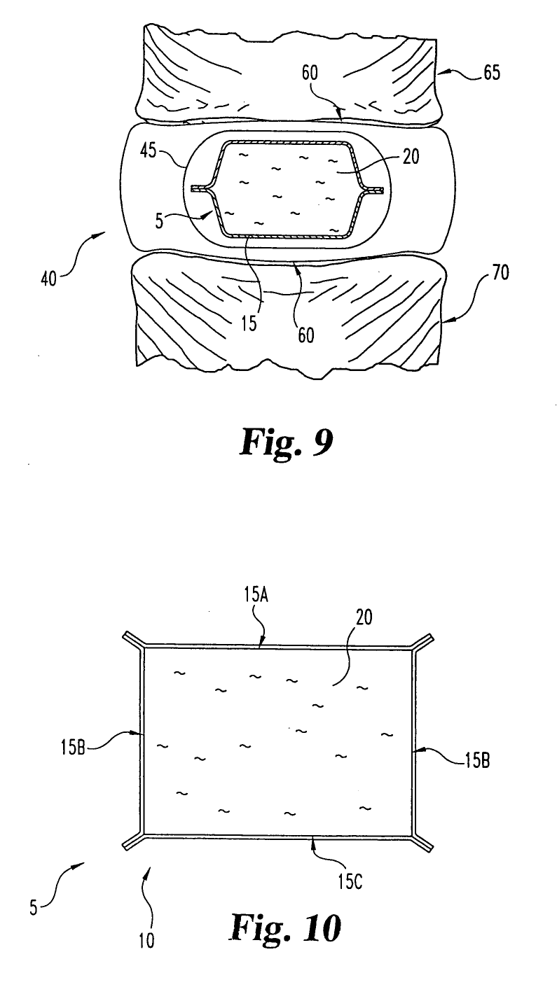

Looking next at FIG. 9, prosthetic nucleus 5 is shown expanded due to the passage of water across the envelope's membrane 15. More particularly, after prosthetic nucleus 5 is deployed in the body, water (which is present in extracellular body fluid) passes through membrane 15 and contacts the at least one solute 20, causing the solute to go into solution, thereby establishing an osmotic engine by which the envelope will inflate and pressurize. The at least one solute 20 contained within envelope 10 may vary between supersaturated and non-saturated, depending on the amount of the at least one solute 20 and water present within envelope 10. In FIG. 9, the end plates 60 of disc 40 have expanded according to the expansion of the envelope, whereby to restore spinal disc 40 to its proper configuration and to hold vertebral bodies 65 and 70 apart.

When forming a prosthetic nucleus for an interverbral disc, it is important to ensure that the prosthetic nucleus (1) reliably assumes a desired configuration, and (2) provides the proper anatomical properties.

More particularly, it is generally desirable that the prosthetic nucleus be constructed so that its expansion takes place primarily in a vertical direction rather than in a radial direction. This is generally desirable to avoid lateral disc bulging which could impinge upon surrounding anatomical structures, e.g., nerves. In addition, it is generally important that the vertical expansion take place to the anatomically appropriate degree. To this end, envelope 10 may be formed with a configuration so as to control the direction and degree of expansion.

Thus, for example, and looking now at FIG. 10, prosthetic nucleus 5 could have its envelope 10 formed out of three separate sections of membrane 15, i.e., a top section 15A, a side section 15B and a bottom section 15C, whereby when envelope 10 is inflated, such as shown in FIG. 10, the prosthetic nucleus will assume a well-defined cylindrical shape (e.g., similar to that of a tunafish can).

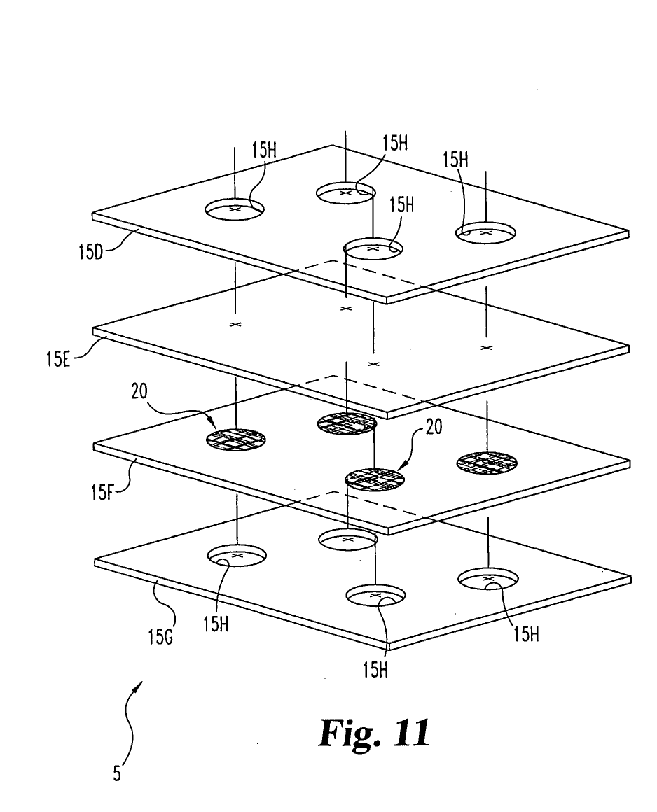

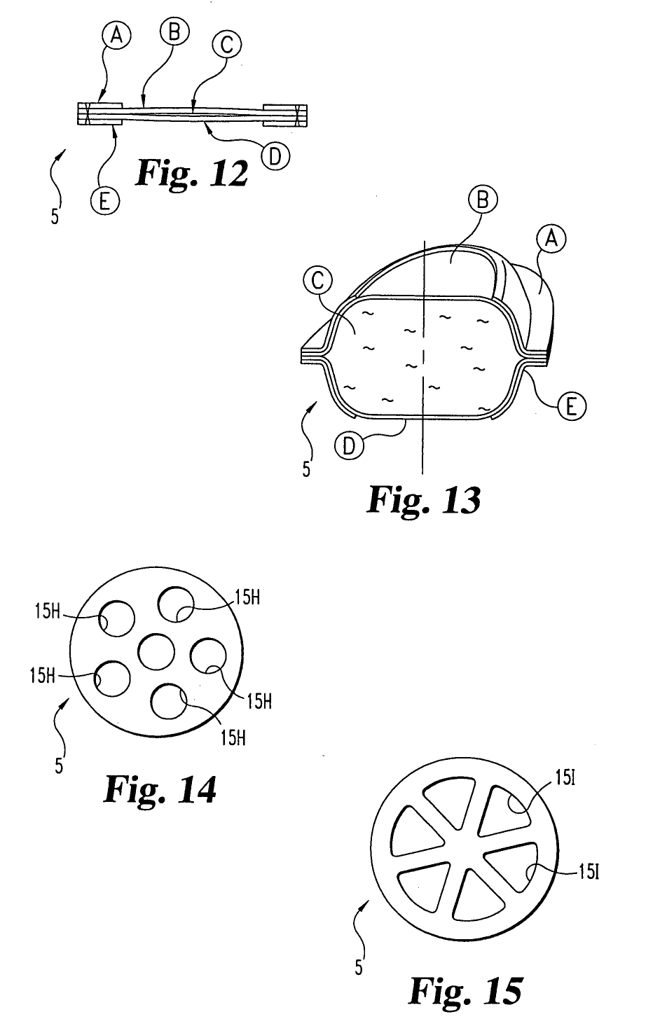

Alternatively, and looking now at FIGS. 11-14, prosthetic nucleus 5 could use a laminated construction to form the nucleus. More particularly, prosthetic nucleus 5 could comprise four sections of membrane, e.g., an upper edge 15D, an upper top membrane 15E, a lower bottom membrane 15F and a lower edge membrane 15G, with the at least one solute 20 (e.g., initially in tablet form) being located between upper top membrane 15E and lower bottom membrane 15F. Upper edge membrane 15D and lower edge membrane 15G have a plurality of circular openings 15H formed therein, whereby prosthetic nucleus 5 will lie substantially flat in its uninflated state (FIG. 12) and will inflate to a desired disc-like shape (FIG. 13).

Alternatively, circular openings 15H (FIG. 14) may be replaced with wedge-shaped openings 15I as shown in FIG. 15, or with openings having some alternative configuration.

It is also possible to form prosthetic nucleus 5 with internal structure so as to control the direction and degree of disc inflation.

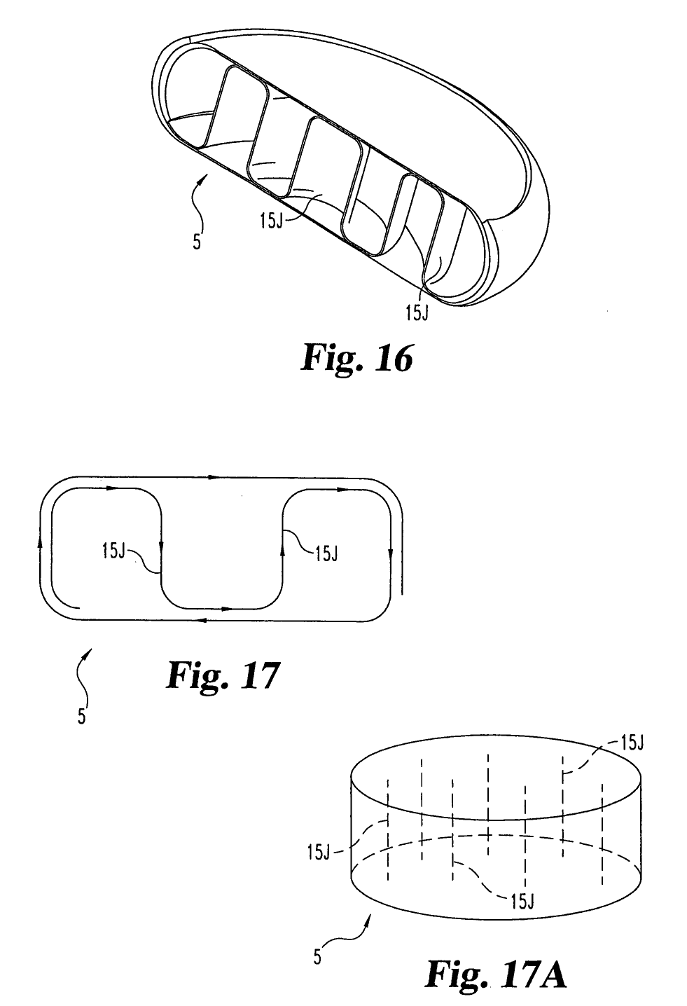

Thus, for example, and looking now at FIG. 16, there is shown a prosthetic nucleus 5 which has a plurality of internal vertical walls 15J which limit the extent of vertical expansion of prosthetic nucleus 5. Vertical walls 15J may be configured so that the interior of the prosthetic nucleus comprises a single chamber, or vertical walls 15J may be configured so as to subdivide the interior of the prosthetic nucleus into a plurality of separate chambers or cells.

Another possible internal vertical wall configuration is shown in FIG. 17.

It is also possible to provide other forms of internal support structure to limit the extent of vertical expansion of prosthetic nucleus 5. Thus, in FIG. 17A there is shown a prosthetic nucleus 5 having a plurality of vertical filaments 15J for limiting the extent of vertical expansion of prosthetic nucleus 5.

As noted above, the force F_{I} generated inside envelope 10 is substantially higher than the external force F_{E} imposed on envelope 10. As a result, the tensile forces F_{V} induced in envelope 10 will be substantial. In this respect, it should be appreciated that aforementioned internal vertical support structures 15J may help provide the tensile forces F_{V} used to help balance the large osmotic forces F_{I} generated within envelope 10.

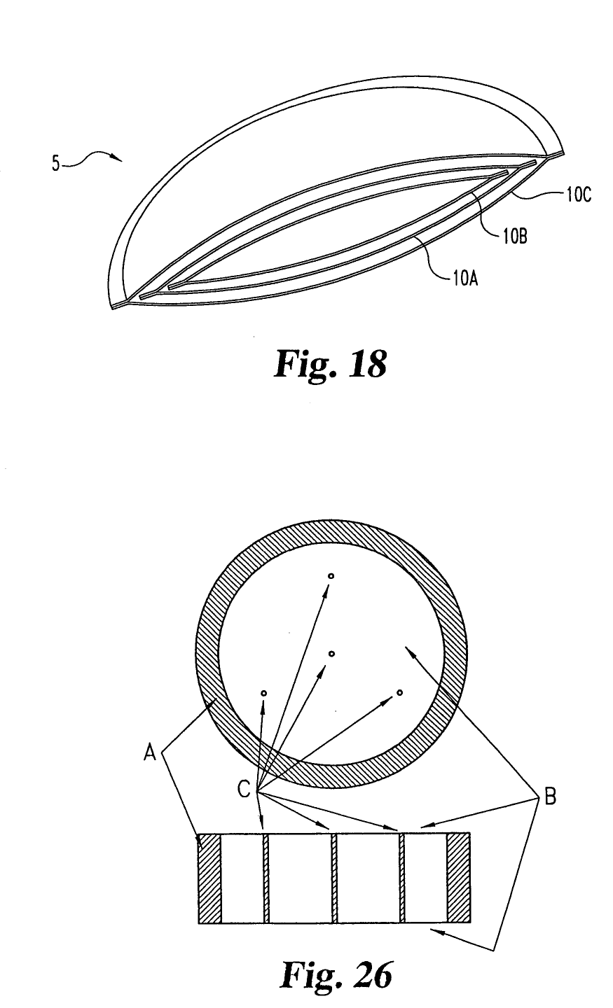

FIG. 18 shows another possible prosthetic nucleus configuration, wherein prosthetic nucleus 5 comprises a plurality of nested envelopes 10A, 10B, 10C, etc.

It is also important that prosthetic nucleus 5 have the proper anatomical properties. For one thing, the prosthetic nucleus 5 should maintain a substantially constant volume in the short term even as the skeletal forces imposed on the prosthetic nucleus change. And the prosthetic nucleus must remain adequately compliant to changing external loads.

To this end, it has been discovered that the load on a typical disc (e.g., the L3 disc) in a typical human (e.g., 154 pounds) is approximately as follows:

| | |
|---|---|
| standing upright | 112 pounds |
| laying supine, awake | 56 pounds |
| bending, lifting, etc. | up to |
| | 472 pounds |

Assuming that the nucleus takes 70% of the compressive load and the annulus takes 30% of the compressive load, the nucleus loading range is from 39 pounds to 330 pounds.

Furthermore, the nucleus typically fills 30-50% of the area of the total disc (annulus plus nucleus), and the total disc area for the L3 disc is approximately 2.1 inch². Therefore, the area of a typical nucleus is between about 0.64 inch² and 1.05 inch².

Assuming moderate loading (upright., long term) of a smaller nucleus, the pressure can be approximated by:
(112 pounds×0.70)/0.64 inch²=123 psi
(123 psi)×1.5=185 psi

As noted above, the at least one solute 20 comprises a material and a quantity sufficient to generate, when the prosthetic nucleus is deployed in the body, an internal force F_{I} which is (1) significantly greater than the external forces F_{E} imposed on the prosthetic nucleus, with the volume of the prosthetic nucleus remaining relatively constant even as the skeletal load on the prosthetic nucleus changes, and (2) low enough that the prosthetic nucleus will remain adequately compliant to changing skeletal loads.

Thus, where
F_{E}=123 psi
and where
F_{I}>>F_{E}
it will be seen that the at least one solute 20 comprises a material and a quantity sufficient to generate, when the prosthetic nucleus pulposus is deployed in the body, an osmotic force significantly higher than 123 psi.

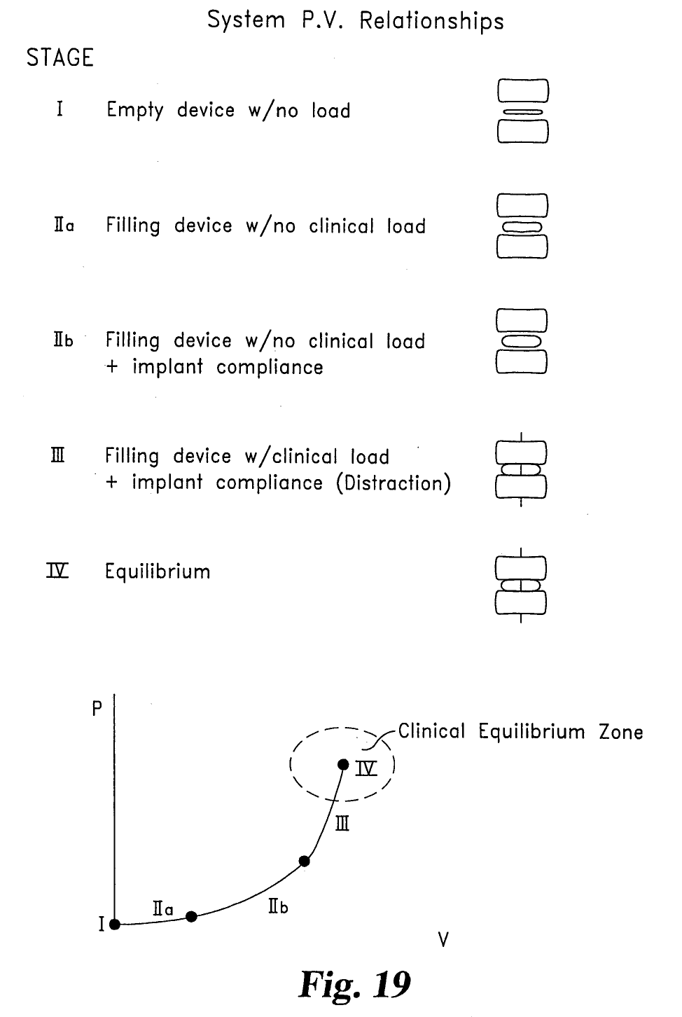

With the osmotic engine of prosthetic nucleus 5, an equilibrium is established according to the load imposed on the nucleus. In particular, and looking next at FIG. 19, the system establishes a pressure-volume (P-V) relationship which eventually stabilizes at an equilibrium condition.

It will also be appreciated that prompt equilibration of an implanted envelope with its surroundings is desirable. As noted above, choices of a single solute or multiple solutes and a complementary, non-permeable membrane can be made to foster prompt equilibration. Even greater speed can be achieved, however, by the use of a supplemental solute of low molecular weight that can actually permeate the membrane used. This supplemental solute will exert its osmotic activity shortly after implantation, increasing the osmotic driving force for water imbibitions above that provided by the primary solute. Since the membrane is not impermeable to the supplemental solute, however, the supplemental solute will ultimately escape from the envelope and will not affect the long-term behavior of the implant.

Prosthetic nucleus 5 is preferably delivered in an uninflated, folded or rolled configuration using a minimally invasive technique. More particularly, prosthetic nucleus 5 may be delivered by folding it up into a reduced cross-section, inserting it into a cannula, placing the cannula into the body so that the distal end of the cannula is positioned into the void 45 created within natural disc 40, and then deployed into the disc, whereupon the prosthetic disc will automatically inflate due to the presence of water present within the disc. See, for example, U.S. patent application Ser. No. 09/559,899, which patent application has been incorporated herein by reference, and which illustrates how this may be done.

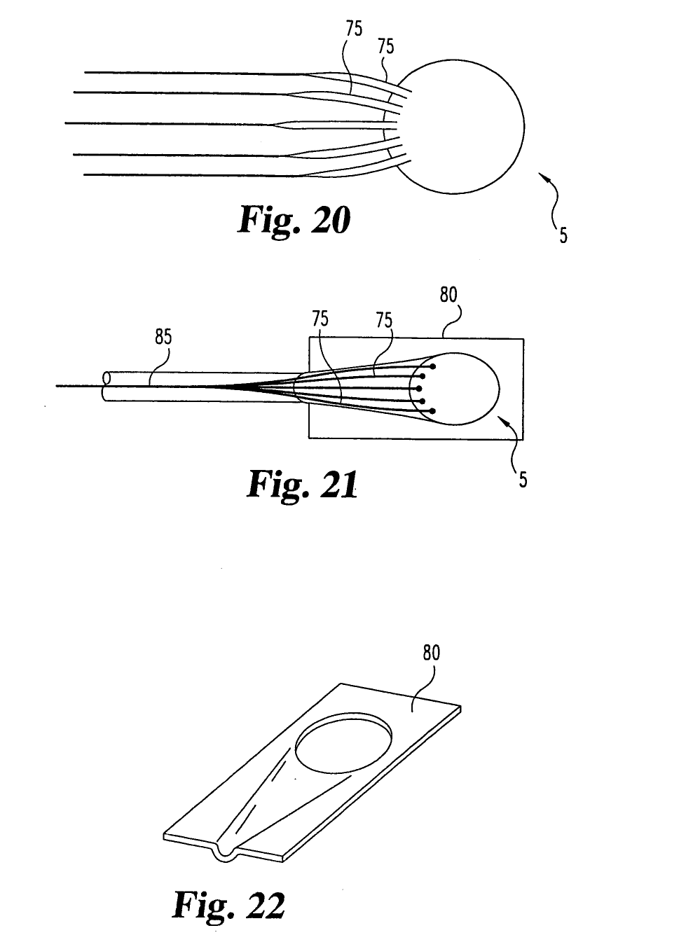

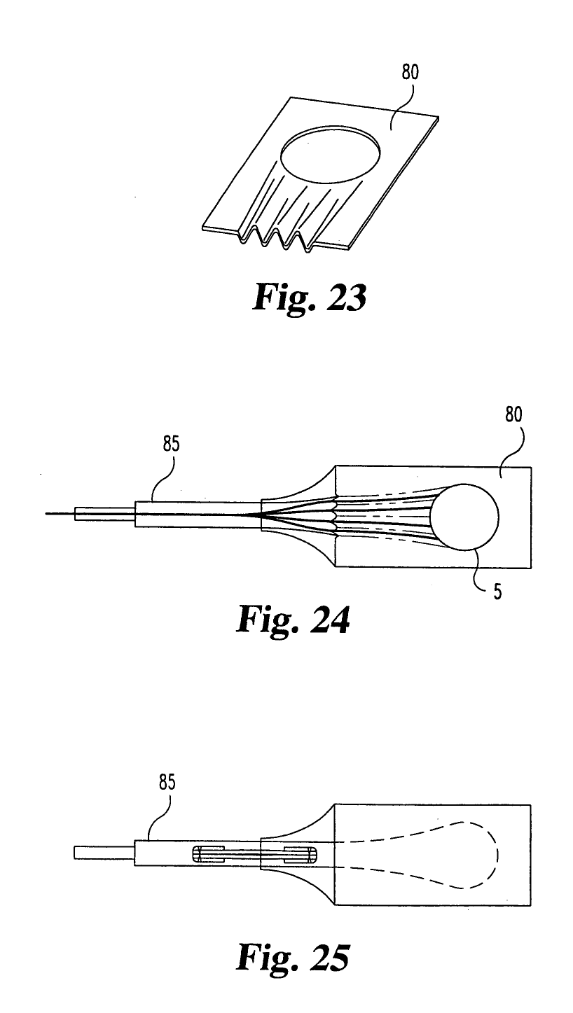

Alternatively, and looking now at FIGS. 20-25, there is shown a technique for loading a prosthetic nucleus 5 into a cannula. In essence, with this technique, a plurality of filaments 75 are attached to the prosthetic nucleus, whereby the nucleus may be drawn through a folding die 80 and thereby loaded into a deployment cannula 85. The prosthetic nucleus may thereafter be ejected from cannula 85 using a plunger (not shown).

The rate of water transport into the prosthetic nucleus is of concern. Water transport may be facilitated by the use of a membrane that is thin, extensive in area, and possesses a high intrinsic permeability to water. Water transport may also be facilitated by making the osmotic driving force as high as possible, consistent with the two opposing criteria: that the solute mass and volume not be excessive, and that the equilibrium osmotic pressure be consistent with the mechanical design of the envelope. These criteria may be relaxed by the use of a supplemental small molecule to which the chosen membrane is somewhat permeable. Inasmuch as the molecule is small, it introduces less mass and volume than would a larger, impermeable molecule. However, the small molecule can permeate the membrane, it will leave the envelope and will not contribute to the equilibrium osmotic pressure. Obviously, a suitable molecule must be at least transiently acceptable in the body fluids surrounding the prosthesis.

In the foregoing discussion, there has been disclosed an envelope 10 for forming a prosthetic nucleus for an intervertebral disc. However, it should also be appreciated that envelope 10 may also be used to form a complete prosthetic intervertebral disc if desired.

It would be appreciated that by carefully designing the overall system (i.e., envelope and solute), the prosthesis can be tailored to biomechanically mimic the natural anatomical structure it is to replace.

### EXAMPLE 1

A particular realization of the invention disclosed herein is considered below. This consideration illustrates the principles on which the invention is based and shows how these principles interact in suitable realizations.

FIG. 26 shows a top and side view of a synthetic nucleus whose envelope comprises a cylindrical ring, A, and a top and bottom piece B composed of membrane material that is permeable to water and impermeable to a solute that is enveloped by the ring and the membrane segments. The apparatus is presumed to have come to equilibrium with the surrounding fluid so that it has an internal hydrostatic pressure equal to the osmotic pressure established by the solute in the enclosed volume. Solid members C, capable of supporting a tensile stress, connect the two membrane segments.

For purposes of illustration, the area of membrane in contact with vertebrae is taken to be 1.5 in² and the compressive force applied to this area by the vertebrae and surrounding tissues is taken to be 450 lb. A pressure of 300 psi within the envelope is required to support this load. Sufficient solute is provided, however, to generate 600 psi of pressure and 900 lb of force. The dimensions and mechanical properties of the load-bearing elements are chosen to counterbalance the remaining 450 lb of force at an envelope height that is anatomically desirable, e.g., 0.25 in. If the load-bearing elements have a Young's modulus of 5,000 psi and an area of 0.6 in², they will be stretched 15% from their unloaded length. If the load is then reduced to 50 lb, the hydrostatic pressure in the envelope will fall below the osmotic pressure and additional water will enter. The entry of water will have two effects: (1) a reduction of the solute concentration and consequently of the osmotic pressure, and (2) an increase in tension within the load-bearing members. The net change in height is about 0.02″, or about 8.4%. Thus it will be seen that the volume of the envelope will remain relatively constant even as the external load imposed on the envelope changes. Furthermore, it will be appreciated that by carefully designing the overall system (i.e., envelope and solute), the prosthesis can be tailored to biomechanically mimic the natural anatomical structure it is to replace. In the absence of the load-bearing elements, the volume change accompanying the large, but possible, change in load would be very large and clinically unacceptable.

More particularly, the applied force. F is opposed by two forces from the prosthesis: (1) the internal hydrostatic pressure, equal at equilibrium to the osmotic pressure, as dictated by the molar concentration of solute, and (2) the opposing stresses provided by the load-bearing elements, which are in tension. Thus:
F=πA₁−YsA₂ (at equilibrium)
where Y is the Young's modulus of the load-bearing elements, s is the strain, i.e. the quotient of elongation, x, by the original length of the elements, x₀, and A₂ the area of the elements. For the quantities stipulated above:
450=π·1.5−Ys₁A₂
For this example we specify YsA₂ equal to 450 lb. Thus we require π to be 600% psi. Using the values specified above, s₁=0.15, the unstressed length of the load-bearing elements is found to be 0.217″.

If the force is reduced to 50 lb, it is necessary to write the first equation above for the new condition:
50={600 ·[(1.15)/(1+S₂)]·1.5}−{5,000·0.6·S₂}
The first term of this equation is the original osmotic pressure reduced by the change in volume of the envelope, multiplied by the contact area. The second term is the opposing force provided by the load-bearing elements. The equation is written in terms of an unknown strain, s₂, for the new situation. When the equation is solved, s₂ is found to be 0.258. The new thickness of the prosthesis is found to be 0.274 in, a 9.4% increase over the original value of 0.25 in. It is clear that different choices for the modulus, Y, and area of the load-bearing elements, A₂, will result in different dimensional changes and that the apparatus may thus be adapted to a wide range of medical needs and preferences.

The illustrative model is provided with structural elements that confine the transverse or radial dimensions of the apparatus essentially to their original value. Thus, no stress need be applied to the annulus, while the device is capable of providing balancing forces, with appropriate dimensional changes, to a wide range of loadings on the spinal column.

1. A prosthetic nucleus pulposus comprising a closed envelope comprising a membrane and containing at least one solute therein, wherein the membrane is permeable to water and impermeable to the at least one solute, and wherein the solute is soluble in water, whereby when the closed envelope is deployed in an environment containing water, the water will pass through the membrane, contacting the at least one solute and causing the at least one solute to go into solution, thereby establishing an osmotic engine by which the envelope will inflate and pressurize, with this inflation continuing until an equilibrium condition is established between the internal and external pressures acting on the envelope, and further wherein, the closed envelope comprises a construction and at least one solute comprises a material and a quantity sufficient to generate internal pressure, when the prosthetic nucleus pulposus is deployed in the body, which is (1) significantly greater than the external pressure imposed on the prosthetic nucleus pulposus by external forces, with the closed envelope being capable of withstanding such internal pressure, with the volume of the prosthetic nucleus pulposus remaining relatively constant even as the external load imposed on the prosthetic nucleus pulposus changes, and (2) low enough that the prosthetic nucleus pulposus will remain adequately compliant to changing external loads by accommodating changing external loads in the short term by an appropriate controlled deformation of the closed envelope.

2. A prosthetic nucleus pulposus according to claim 1 wherein said envelope is formed substantially entirely out of said membrane.

3. A prosthetic nucleus pulposus according to claim 2 wherein said envelope includes a reinforcing mesh.

4. A prosthetic nucleus pulposus according to claim 3 wherein said reinforcing mesh is positioned internal to said membrane.

5. A prosthetic nucleus pulposus according to claim 3 wherein said reinforcing mesh is positioned external to said membrane.

6. A prosthetic nucleus pulposus according to claim 3 wherein said reinforcing mesh is contained within said membrane.

7. A prosthetic nucleus pulposus according to claim 1 wherein said membrane comprises a window formed in a wall of said envelope.

8. A prosthetic nucleus according to claim 1 wherein said membrane comprises a homogenous membrane with suitable water permeable characteristics.

9. A prosthetic nucleus according to claim 1 wherein said membrane comprises a polyurethane block copolymer with hydrophilic segments.

10. A prosthetic nucleus pulposus according to claim 1 wherein said membrane comprises at least one of the group consisting of cellulose acetate, cellulose acetate butyrate, cellulose nitrate, crosslinked polyvinyl alcohol, polyurethanes, nylon 6, nylon 6.6, aromatic nylon, polyvinyl acetate, plasticized polyvinyl acetate, polyvinyl butyrate, and ethylene vinyl acetate copolymers.

11. A prosthetic nucleus pulposus according to claim 1 wherein the membrane has a thickness of between about 0.010 and 0.030 inch.

12. A prosthetic nucleus pulposus according to claim 1 wherein said envelope has a disc-like shape.

13. A prosthetic nucleus pulposus according to claim 1 wherein said at least one solute comprises a solid when it is placed into said evelope.

14. A prosthetic nucleus pulposus according to claim 1 wherein said at least one solute comprises a paste when placed into said envelope.

15. A prosthetic nucleus pulposus according to claim 1 wherein said at least one solute comprises a liquid concentrate when placed into said envelope.

16. A prosthetic nucleus pulposus according to claim 1 wherein said at least one solute is placed in said envelope before the prosthetic nucleus pulposus is placed in the body.

17. A prosthetic nucleus pulposus according to claim 1 wherein said at least one solute is placed in said envelope after the prosthetic nucleus pulposus is placed in the body.

18. A prosthetic nucleus according to claim 1 wherein said at least one solute comprises polyacrylamide.

19. A prosthetic nucleus pulposus according to claim 1 wherein said at least one solute comprises at least one of the group consisting of sodium chloride, calcium chloride, magnesium chloride, magnesium sulfate, potassium sulfate, potassium chloride, sodium sulfate, sodium acetate, ammonium phosphate, ammonium sulphate, calcium lactate and magnesium succinate.

20. A prosthetic nucleus pulposus according to claim 1 wherein said at least one solute comprises at least one of the group consisting of sucrose, glucose, fructose, glycine, alanine, valine and vinyl pyrrolidone.

21. A prosthetic nucleus pulposus according to claim 1 wherein said at least one solute comprises at least one of the group consisting of poly-n-vinylpyrrolidone, carboxymethylcellulose and polyethylene glycols.

22. A prosthetic nucleus pulposus according to claim 1 wherein said at least one solute comprises at least one of the group consisting of manitol, urea, blood byproducts, proteins and dextran.

23. A prosthetic nucleus pulposus according to claim 1 wherein said envelope is formed out of a top section, a side section and a bottom section, whereby to control the direction and degree of envelope expansion.

24. A prosthetic nucleus pulposus according to claim 1 wherein said envelope is formed out of an upper edge section, an upper top section, a lower bottom section and a lower edge section, with said solute being located between said upper top section and said lower bottom section and further wherein said upper edge section and said lower edge section comprise openings therein, whereby to control the direction and degree of envelope expansion.

25. A prosthetic nucleus pulposus according to claim 21 wherein said openings are circular.

26. A prosthetic nucleus pulposus according to claim 24 wherein said openings are wedge-shaped.

27. A prosthetic nucleus pulposus according to claim 1 wherein said envelope comprises at least one internal wall, whereby to control the direction and degree of envelope expansion.

28. A prosthetic nucleus pulposus according to claim 27 wherein said at least one wall subdivides the interior of the prosthetic nucleus into a plurality of separate chambers.

29. A prosthetic nucleus pulposus according to claim 1 wherein said prosthetic nucleus pulposus comprises a plurality of nested envelopes.

30. A prosthetic nucleus pulposus according to claim 1 wherein said osmotic pressure is greater than 100 psi.

31. A prosthetic nucleus according to claim 1 wherein said at least one solute comprises a plurality of solutes.

32. A prosthetic nucleus according to claim 31 wherein said plurality of solutes comprise a first solute, and a second solute, and wherein said membrane is permeable to said second solute.

33. A method for replacing the nucleus pulposus of an intervertebral disc, comprising the steps of:
providing a prosthetic nucleus pulposus comprising a closed envelope comprising a membrane and containing at least one solute therein, and wherein the membrane is permeable to water and impermeable to the at least one solute, and wherein the at least one solute is soluble in water, whereby when the closed envelope is deployed in an environment containing water, the water will pass through the membrane, contacting the at least one solute and causing the at least one solute to go into solution, thereby establishing an osmotic engine by which the envelope will inflate and pressurize, with this inflation continuing until an equilibrium condition is established between the internal and external pressures acting on the envelope, and further wherein the closed envelope comprises a construction and at least one solute comprises a material and a quantity sufficient to generate internal pressure, when the prosthetic nucleus pulposus is deployed in the body, which is (1) significantly greater than the external pressure imposed on the prosthetic nucleus pulposus by external forces, with the closed envelope being capable of withstanding such internal pressure, with the volume of the prosthetic nucleus pulposus remaining relatively constant even as the external forces on the prosthetic nucleus pulposus changes, and (2) low enough that the prosthetic nucleus pulposus will remain adequately compliant to changing external loads by accommodating changing external loads in the short term by an appropriate controlled deformation of the closed envelope;
creating a void in the natural nucleus pulposus of an intervertebral disc; and
deploying the prosthetic nucleus pulposus in the void in the intervertebral disc.

34. A prosthetic intervertebral disc comprising a closed envelope comprising a membrane and containing at least one solute therein, wherein the membrane is permeable to water and impermeable to the at least one solute, and wherein the at least one solute is soluble in water, whereby when the closed envelope is deployed in an environment containing water, the water will pass through the membrane, contacting the at least one solute and causing the at least one solute to go into solution, thereby establishing an osmotic engine by which the envelope will inflate and pressurize, with inflation continuing until an equilibrium condition is established between the internal and external pressures acting on the envelope, and further wherein the closed envelope comprises a construction and the at least one solute comprises a material and a quantity sufficient to generate an internal pressure, when the prosthetic intervertebral disc is deployed in the body, which is (1) significantly greater than the external pressure imposed on the prosthetic intervertebral disc by external forces, with the closed envelope being capable of withstanding such internal pressure, with the volume of the prosthetic intervertebral disc remaining relatively constant even as the external load imposed on the prosthetic intervertebral disc changes, and (2) low enough that the prosthetic intervertebral disc will remain adequately compliant to changing external loads by accommodating changing external loads in the short term by an appropriate controlled deformation of the closed envelope.

35. A method for replacing an intervertebral disc, comprising the steps of:
providing a prosthetic intervertebral disc comprising a closed envelope comprising a membrane and containing at least one solute therein, wherein the membrane is permeable to water and impermeable to the at least one solute, and wherein the at least one solute is soluble in water, whereby when the closed envelope is deployed in an environment containing water, the water will pass through the membrane, contacting the at least one solute and causing the at least one solute to go into solution, thereby establishing an osmotic engine by which the envelope will inflate and pressurize, with this inflation continuing until an equilibrium condition is established between the internal and external pressures acting on the envelope, and further wherein the closed envelope comprises a construction and the at least one solute comprises a material and a quantity sufficient to generate an internal pressure, when the prosthetic intervertebral disc is deployed in the body, which is (1) significantly greater than the external pressure imposed on the prosthetic intervertebral disc by external forces, with the closed envelope being capable of withstanding such internal pressure, with the volume of the prosthetic intervertebral disc remaining relatively constant even as the external load imposed on the prosthetic intervertebral disc changes, and (2) low enough that the prosthetic intervertebral disc will remain adequately compliant to changing external loads by accommodating changing external loads in the short term by an appropriate controlled deformation of the closed envelope;
removing the natural intervertebral disc; and
deploying the prosthetic intervertebral disc in the void left by the removal of the natural intervertebral disc.

36. A prosthetic nucleus pulposus according to claim 1 wherein said envelope contains a supplemental solute and further wherein said membrane is not impermeable to said supplemental solute.
